(19) United States
(12) Patent Application Publication
(10) Pub. No.: US 2025/0281606 A1
(43) Pub. Date: Sep. 11, 2025

(54) CANNABIDIOL FOR AUGMENTING VACCINE MEDIATED IMMUNITY AND PROPHYLAXIS OF COVID-19

(71) Applicants: Shreema MERCHANT, Surrey, British Columbia (CA); Manit PATEL, Surrey, British Columbia (CA); AKSEERA PHARMA CORP., Vancouver, British Columbia (CA)

(72) Inventor: Shreema MERCHANT, Surrey, British Columbia (CA)

(21) Appl. No.: 18/266,655

(22) PCT Filed: Feb. 14, 2022
(86) PCT No.: PCT/IB2022/051304

§ 371 Date: Jun. 12, 2023

(30) Foreign Application Priority Data
Dec. 12, 2020 (IN) 202021054151

(51) Int. Cl. A61K 39/39; A61K 31/00; A61K 39/00; A61K 39/215; A61P 31/14

(52) U.S. Cl. CPC A61K 39/39; A61K 31/658; A61K 39/215; A61P 31/14; A61K 2039/55511; A61K 2039/575

(57) ABSTRACT

The present invention relates to a pharmaceutical composition comprising therapeutically effective amount of Cannabidiol for administration with a Covid-19 vaccine to a mammal/human to sustain and/or enhance effect of vaccine. Further the invention relates to methods to sustain and/or enhance effect of a Covid-19 vaccine in a mammal/human by administering to such a mammal/human a pharmaceutical composition comprising a therapeutically effective amount of Cannabidiol with a Covid-19 vaccine.

Administration of Cannabidiol with vaccine can be of following types: i) before administering Covid-19 vaccine; or ii) along with Covid-19 vaccine; or iii) after administering Covid-19 vaccine; or iv) any combination of i, ii and iii including before, along with and after administering Covid-19 vaccine.

### FIELD OF THE INVENTION

The present invention evaluates if vaccine efficacy can be sustained and whether it can be enhanced over time by combining in a mammal or human vaccine administration with administration of one or more Cannabinoids, particularly Cannabidiol in a suitable form. The purpose is to check said effects and propose potentiating effects of a pharmaceutical composition containing therapeutically effective amount of Cannabidiol (CBD) when such composition is administered with vaccine.

Administration with vaccine can be of following types:

i) before administering Covid-19 vaccine; or

ii) along with Covid-19 vaccine; or

iii) after administering Covid-19 vaccine; or

iv) any combination of i, ii and iii including before, along with and after administering Covid-19 vaccine.

### BACKGROUND OF THE INVENTION

Treatment or prophylaxis of Covid-19 infectious disease is extremely challenging. This is more so because SARS-COV-2 has many variants and some of the variants have i) increased transmissibility, ii) increased virulence, and iii) reduced effectiveness of vaccines.

Covid-19 vaccine effectiveness decreases over time and a second dose or a subsequent dose is required and shall be administered as per the schedule. This decrease is commonly known as “Waning effect” or “Waning of Vaccine”. The overall decrease in efficacy is also due to new SARS-COV-2 variants which are formed incessantly in various geographies. The Alpha variant, B.1.1.7, was first found in the United Kingdom in September 2020, as per WHO. The Beta variant, B.1.351, was found in South Africa in May 2020. The next highly transmissible variant, Gamma, P.1, was found in Brazil in November 2020. The fourth variant, Delta, B.1.617.2, or the super-Alpha, as researchers call it, was identified in India in October 2020. The Omicron, B.1.1.529, variant was traced in November 2021. Compared with other variants, Omicron contains more mutations, in the spike that recognizes host cells, thus accounting for its transmissibility.

Over two hundred vaccines are under development against the SARS-COV-2 virus (Patel M, 2020) due to the high mortality rates and the global reach of infection.

Bharat Biotech's Covid-19 Vaccine (COVAXIN®) is an inactivated (killed) vaccine developed using Whole-Virion Inactivated Vero Cell derived platform technology. Inactivated vaccines contain dead virus and do not replicate.

Developed by the Jenner Institute, University of Oxford, ChAdOx 1 is an adenoviral vector for vaccines wherein chimpanzee adenovirus is modified into nonreplicating viral vector.

BNT162b2 is a nucleoside-modified RNA vaccine formulated as a lipid nanoparticle.

In an article titled “Spike-antibody waning after second dose of BNT162b2 or ChAdOx1” published in Lancet on Jul. 15, 2021, Shrotri M et al mentions as follows:

“A significant trend of declining S-antibody levels was seen with time for both ChAdOx1 (p<0.001) and BNT162b2 (p<0.001; figure; appendix), with levels reducing by about five-fold for ChAdOx1, and by about two-fold for BNT162b2, between 21-41 days and 70 days or more after the second dose. This trend remained consistent when results were stratified by sex, age, and clinical vulnerability (appendix). For BNT162b2, S-antibody levels reduced from a median of 7506 U/mL (IQR 4925-11 950) at 21-41 days, to 3320 U/mL (1566-4433) at 70 or more days. For ChAdOx1, S-antibody levels reduced from a median of 1201 U/mL (IQR 609-1865) at 0-20 days to 190 U/mL (67-644) at 70 or more days.”

Typically, at an interval of 4-12 weeks, another dose of vaccine becomes essential. It is interesting to note that even after administration of vaccine, the levels of antibodies viz. IgG and IgM do not rise immediately. Insufficient, inadequate and inconsistent levels cannot provide a complete protection immediately after vaccination and in between two vaccine doses. This is more conspicuously observed during third wave of infection where vaccinated population is also largely affected.

Thus, there is a need in the art to provide an adjuvant therapy along with vaccines that can potentiate effects of vaccines by sustaining and/or enhancing levels of

IgG and IgM antibodies.

### SUMMARY OF THE INVENTION

Under the first aspect, the invention provides a combined administration of a Covid-19 vaccine and a pharmaceutical composition comprising therapeutically effective amount of Cannabidiol to evaluate whether such combination sustains or enhances effects of said vaccine in a mammal or human.

Under the second aspect, the invention provides a combined administration of a Covid-19 vaccine and a pharmaceutical composition comprising therapeutically effective amount of Cannabidiol to evaluate effect of such combination wherein the composition is administered before administration of vaccine and also alternatively both before, along and after administration of vaccine.

Under the third aspect, the invention provides a combined administration of a Covid-19 vaccine and a pharmaceutical composition comprising therapeutically effective amount of Cannabidiol to evaluate effect of such combination wherein at least two different types of vaccines are employed and the effects produced therein are compared.

In all above aspects, effect of combination is compared with negative control (no vaccine and no composition) and vehicle control (only vaccine, no composition).

In yet another approach invention provides kits having one or more vaccine and pharmaceutical compositions of Cannabidiol for administration before or along with or after administration of said vaccine to a mammal or a human.

### BRIEF DESCRIPTION OF FIGURES

FIG. 1 provides a proposed study designed for studying effects of Cannabidiol on vaccine efficacy.

FIGS. 2A-2L provide Antibody titre responses of male and female mice for Covishield vaccine.

FIGS. 2A and 2B respectively provide IgG (FIG. 2A) and IgM (FIG. 2B) antibody titres measured at various time points.

FIGS. 2C and 2D show the average overall IgG response (FIG. 2C) and IgM response (FIG. 2D) over 28 days.

FIGS. 2E and 2F-FIGS. 2E and 2F show that at any time point measured, mice injected with vaccine, but not given CBD, failed to have a significant antibody response compared to mice not given vaccine.

FIGS. 2G and 2H provide IgG (FIG. 2G) and IgM (FIG. 2H) antibody titres measured at various timepoints are shown.

FIGS. 2I and 2J provide area under the curves (AUC) for each mouse and graphed to show the average overall IgG response (FIG. 2I) and IgM response (FIG. 2J) over 28 days.

FIGS. 2K and 2L provide that at any time point measured, mice injected with vaccine, but not given CBD, failed to have a significant antibody response compared to mice not given vaccine.

FIGS. 3A to 3J provide data for Covaxin.

FIGS. 3A and 3B provide that Vaccination of mice with Covaxin caused an increase in IgG and IgM responses, either when given alone or when given with CBD continuously, or CBD given prior to injection (and discontinued at injection), compared to mice that were not vaccinated.

### DETAILED DESCRIPTION OF THE INVENTION

Wide range of disease severities, including the presence of asymptomatic carriers, poses a major challenge in controlling the pandemic through targeted treatment alone. With an immense possibility opening up for using Non-Psychotropic Cannabinoids for augmenting the cellular anti-viral defenses, which could help in a prophylactic manner and also because these cannabinoids have a unique property to act as a modulator, the inventors further propose to administer Cannabinoids in patients suffering from Covid-19 for its modulating action and also to healthy individuals for its prophylactic action. This further unfolds application of Non-Psychotropic Cannabinoids along with Covid-19 vaccines.

Over two hundred vaccines are under development against the SARS-COV-2 virus (Patel M, 2020) due to the high mortality rates and the global reach of infection. Vaccine development for COVID-19 can be classified into six major strategies using live attenuated virus, virus-like particles, inactivated virus, nucleic acid-based, recombinant viral vectored, and protein subunits.

The vaccines can be categorized based on
1. The whole-microbe approach;
2. The subunit approach; and
3. The genetic approach.

The vaccines can also be categorized based on a component viral vaccine or whole virus vaccine.

The Component Viral Vaccines comprise of the following types:

1. Protein Subunit: containing isolated and purified viral proteins

2. Virus-like Particles (VLP): containing viral proteins that mimic the structure of the virus, but no genetic material

3. DNA-based and RNA-based: containing viral genetic material (such as mRNA) which provides the instructions for making viral proteins

4. Non-Replicated Viral Vector: containing viral genetic material packaged inside another harmless virus that cannot copy itself

5. Replicating Viral Vector: containing viral genetic material packaged inside another harmless virus that can copy itself

The Whole Virus Vaccines comprise of the following types:

1. Inactivated: Contains copies of the virus that have been killed (inactivated)

2. Live-Attenuated: Contains copies of the virus that have been weakened (attenuated).

In the present study, following vaccines are employed.
1. Bharat Biotech's Covid-19 Vaccine (COVAXIN®) which is an inactivated (killed) vaccine developed using Whole-Virion Inactivated Vero Cell derived platform technology. Inactivated vaccines contain dead virus and do not replicate.
2. Developed by the Jenner Institute, University of Oxford, ChAdOx1 is an adenoviral vector for vaccines wherein chimpanzee adenovirus is modified into nonreplicating viral vector.

Alternatively, other vaccines such as BNT162b2 which is a nucleoside-modified RNA vaccine formulated as a lipid nanoparticle.

Among the above mentioned vaccines, only the attenuated virus strategy is anticipated to offer a single-dose administration while all the others would need secondary dosage to ensure protection of the host (Jeyanathan et. al. 2020).

Under various aspects, the invention provides
i) a combined administration of a Covid-19 vaccine and a pharmaceutical composition comprising therapeutically effective amount of Cannabidiol to evaluate whether such combination sustains or enhances effects of said vaccine in a mammal or human.
ii) a combined administration of a Covid-19 vaccine and a pharmaceutical composition comprising therapeutically effective amount of Cannabidiol to evaluate effect of such combination wherein the composition is administered before administration of vaccine and also alternatively both before, along and after administration of vaccine;
iii) a combined administration of a Covid-19 vaccine and a pharmaceutical composition comprising therapeutically effective amount of Cannabidiol to evaluate effect of such combination wherein at least two different types of vaccines are employed and the effects produced therein are compared;
iv) kits having one or more vaccine and pharmaceutical compositions of Cannabidiol for administration before or along with or after administration of said vaccine to a mammal or a human.

In all above aspects, effect of combination is compared with negative control (no vaccine and no composition) and vehicle control (only vaccine, no composition). Administration of Cannabidiol with vaccine can be any of the following types:

i) before administering Covid-19 vaccine; or

ii) along with Covid-19 vaccine; or

iii) after administering Covid-19 vaccine; or

iv) any combination of i, ii and iii including before, along with and after administering Covid-19 vaccine.

The present invention provides a pharmaceutical composition of Cannabidiol for administration along with a Covid-19 vaccine to a mammal/human to sustain and/or enhance effect of vaccine and methods to sustain and/or enhance effect of a

Covid-19 vaccine in a mammal/human comprising administering to such a mammal/human a pharmaceutical composition comprising a therapeutically effective amount of Cannabidiol along with a Covid-19 vaccine. In this, the sustenance or enhancement of effect of Covid-19 vaccine comprises maintenance or enhancement of response/level of one or more antibodies selected from IgG and IgM.

Cannabidiol when administered prophylactically, enhances the various vaccine-variants potential (more robust response) against all current and future variants of pathogenic or virulent viruses. The various vaccine-variants are not restricted to single source, unitary-dose or scheduled multi-dose; but also encompass multi-source variants unitary-dose or scheduled multi-dose or mixed dose in combinations

In the present invention, any type of vaccine or a combination of two or more vaccines can be employed. Few countries have adopted mixed vaccines approach. The present invention is also applicable to mixed vaccines where two or more vaccines can be used. For an example, if a combination of mRNA+recombinant+attenuated virus vaccine is employed, the compositions of Cannabidiol would enhance the effect of such mixed vaccines.

Since incessantly new variants are SARS-Cov-2 are encountered, mixed vaccine approach may be essential in future. In a mixed vaccines, two or more vaccines can be employed wherein each vaccine can be employed from 0.001 to 99.998%.

The prominent variants of SARS-Cov-2 include following variants.

The Alpha variant, B.1.1.7, was first found in the United Kingdom in September 2020, as per WHO. The Beta variant, B.1.351, was found in South Africa in May 2020. The next highly transmissible variant, Gamma, P.1, was found in Brazil in November 2020. The fourth variant, Delta, B.1.617.2, or the super-Alpha, as researchers call it, was identified in India in October 2020. The Omicron, B.1.1.529, variant was traced in November 2021. Compared with other variants, Omicron contains more mutations, in the spike that recognizes host cells, thus accounting for its transmissibility.

Cannabidiol (CBD) enhances the immunogenic response to COVID-19 vaccines, although the time of the action may vary with the type of vaccine employed.

However, since attenuated and inactivated viruses can pose a threat of recombination (Tao et. al. 2017) with wildtype strains, causing infection, rather than inducing immunity, additional strategies are favoured, despite the need for repeated injections.

These are only some of the challenges faced with vaccines being developed for COVID-19 infections, as the time frame for the process is squeezed into 1-2 years which would otherwise extend to 10-15 years (Lurie et. al., 2020). Thus, the long term side effects, immunity across serotypes of virus, and impact on susceptible populations, are only some of the critical questions that are associated with vaccine efficacy and safety that will remain unanswered, even as Sars-COV-2 vaccine administration begins in countries most affected by the pandemic.

Cannabidiol and other non-psychotropic Cannabinoids (NPC) either alone or in all possible combinations thereof can play a critical role in reducing symptoms induced by some vaccine strategies, and buy time for the body to induce humoral defense.

Cardiac arrythmias are some of the fatal consequences of SARS-COV-2 infections, and its treatment and the CBD induces cardioprotective effects (Fauda et al, 2020). Therefore CBD may have similar benefits should such side effects be associated with any of the vaccine strategies being investigated for Sars-COV-2 infections.

The ability of attenuated viruses recombining (Cyranoski D, 2020) with wildtype may be a major pitfall associated with attenuated virus vaccine strategy, which may induce a disease state instead of immunity. Should such a recombination event occur, NPCs may prevent uncontrolled SARS-COV-2 replication early on in the infection resulting from innate immune suppression (Remy et al 2020), (Blanco-Melo, 2020). Therefore, they may prevent a dysregulated inflammatory response, and prevent and/or reduce the onset of symptoms. Such preventative strategies involving repurposed molecules like NPCs showing therapeutic value against COVID are discernible adjuncts to enhance vaccine safety and efficacy.

The vaccines clinical trial data for aged population and children can be sparse. The inventors anticipate that the use of NPCs, especially CBD, with vaccines may help improve vaccination outcomes for susceptible patient groups like seniors, patients with underlying conditions and in children who typically are not participating in the clinical trials.

Antibody-dependent enhancement (ADE) is induced in viral infections like dengue due to sub neutralizing concentrations of cross reacting antibodies. However, whether high concentrations of neutralizing antibodies have been associated with the induction of Antibody-dependent enhancement (ADE) is being debated, not only in SARS-COV-2 infections but also in some SARS-COV-2 vaccine strategies, where ADE appears to be induced in animal models. An efficacious vaccine would therefore be one that induces sufficient titers of neutralizing antibodies which most likely will need to be boosted through subsequent administrations. Further in dengue, T-cell mediated antiviral response can reduce the ADE of disease. NPCs that appear to upregulate interferon mediated T cell activation may play a significant role in inducing simultaneous T-cell activation while vaccines and their booster doses help develop humoral immunity (Jeyanathan 2020). The inventors expect the potential candidate NPCs to find optimal use in the interim period between first and booster doses of the vaccines. They anticipate further that the candidate NPCs may improve efficacy and safety of SARS-COV-2 prevention strategies including vaccines.

Thus, inventors propose administering of non-psychotropic Cannabinoids either alone or in all possible combinations thereof for prophylaxis of Covid-19. They can be administered alone, or they can be administered along with vaccine. Such co-administration is expected to enhance vaccine safety and efficacy. The administration can be done simultaneously or sequentially with vaccines or both simultaneously and sequentially with vaccines. The co-administration of non-psychotropic Cannabinoids may differ for different types of vaccines. Clinical trials are proposed for i) administration of non-psychotropic Cannabinoids particularly CBD and ii) co-administration of non-psychotropic Cannabinoids, particularly cannabidiol and vaccines for prophylaxis of Covid-19 and protocols are being prepared based on how vaccines are administered.

Non-psychotropic Cannabinoids which are either administered alone or co-administered along with vaccine will be administered in suitable pharmaceutical formulations/compositions discussed below.

A suitable dose of one or more cannabinoids is from 0.00001 mg/kg of body weight to 4000 mg/kg of body weight for each cannabinoid. The suitable dose can also be 0.00001 to 1000 mg/kg of body weight or 0.00001 to 500 mg/kg of body weight. The preferred dose can be 0.00001 to 100 mg/kg of body weight or from 0.00001 to 10 mg/kg of body weight.

The pharmaceutical compositions of Cannabidiol are previously described in PCT International Applications published as WO2021199078, WO2022018754, WO2021165992 and Indian Patent Application IN202021054151.

The dose will depend on the nature and status of human or animal patient health. It will also depend on age and comorbidities if any. Further, dose will depend on type of composition for example, whether oral or parenteral or topical.

Thus, it is essential to have an alternative treatment which can boost immunity in population before, after and in between Covid vaccine doses whenever inadequate antibodies titer are expected. While many immunity boosting compositions are reported in recent past, their effect and role before, after and during vaccine administration and particularly on levels of IgG and IgM have not been established.

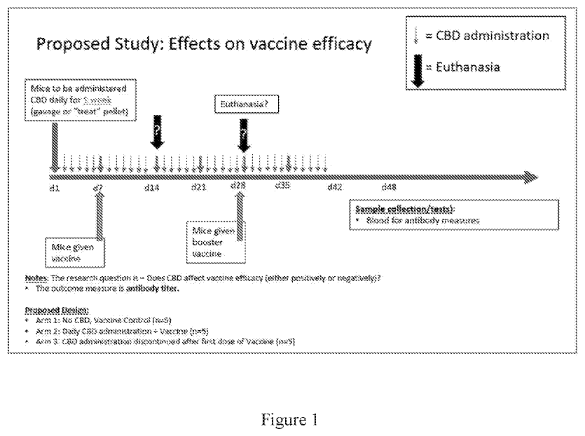

In recent past, Cannabinoids and particularly Cannabidiol has shown promising role in priming, prophylaxis and treatment of Covid-19 infection (WO2021199078 and WO2022018754). It was further proposed (Indian Patent Application IN202021054151) to check effect of Cannabidiol on administration of vaccines. Accordingly, a first proposed study was designed as provided in FIG. 1 to check if Cannabidiol exerts potentiation (enhancement) of effects of vaccines or at least sustains effect of vaccines.

The present invention thus aims to evaluate if vaccine efficacy can be sustained and whether it can be enhanced over time by combining vaccine administration with administration of one or more Cannabinoids, particularly Cannabidiol. The objective of the present study was to evaluate the pharmacological potentiating effect of Cannabidiol (CBD) on Vaccine to enhance the immunity profile of the host compared to vaccine alone. The study is conducted in Balb/c mice.

Following general protocol and conditions are adopted for the study.

### General Protocol And Conditions

8-10 week old female Balb/c mice (21-25 g) are used. Three mice of each sex were used per group. The detailed conditions are provided under example 1.

a). Conditions: The animals are housed under standard laboratory conditions, airconditioned with adequate fresh air supply;

b). Housing: Five animals are housed per cage in standard polypropylene cages having the size: Length 43.0×Breadth 27.0×Height 15.0 cm.

c). Acclimatization: The animals are acclimatized for a period of five days before the start of the study.

d). Diet: The animals are fed ad libitum throughout the acclimatization and study period.

e) Water: Clean uncontaminated drinking water is provided ad libitum throughout the acclimatization and study period.

f). Bedding Material: Clean and sieved paddy husk is used as bedding material for the present study.

g) Test Items: The test items formulations were prepared afresh before dosing.

The mice were divided into following four groups.

1. Negative control (no vaccine, no CBD), 2. Vehicle control—(only vaccine, no CBD),

3. CBD daily from −7 (7 days before vaccine) to 28 days, 4. CBD daily for 7 days before vaccine and discontinued after administration of vaccine.

Blood samples were taken at day 0 (i.e. on the day of vaccine administration), and on 7, 14, 21, and 28 and IgG and IgM levels were measured.

The details are provided under example 2.

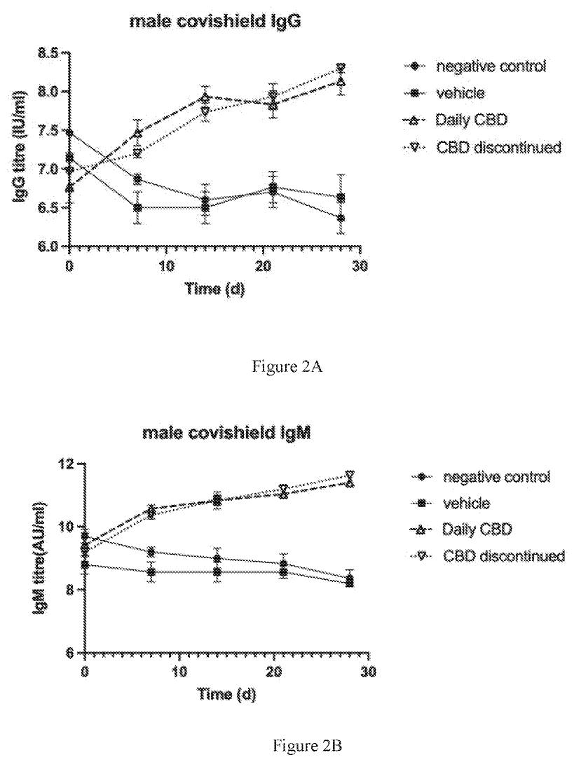

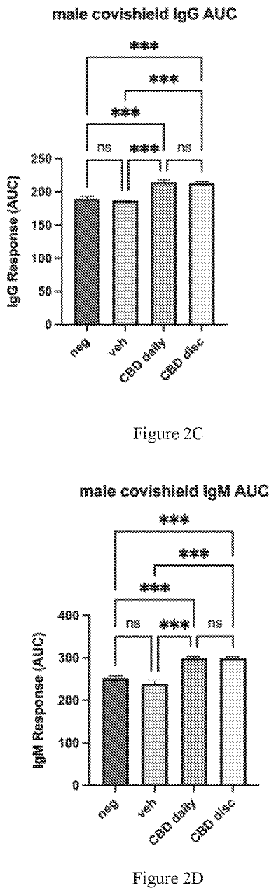

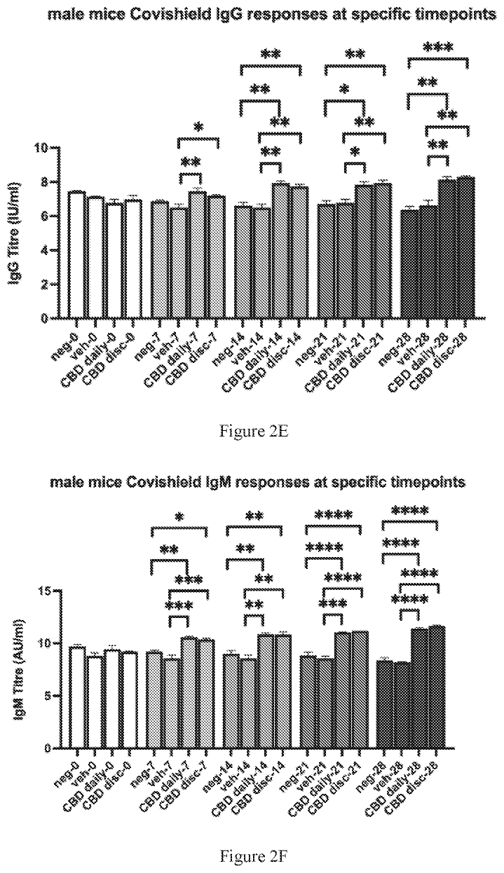

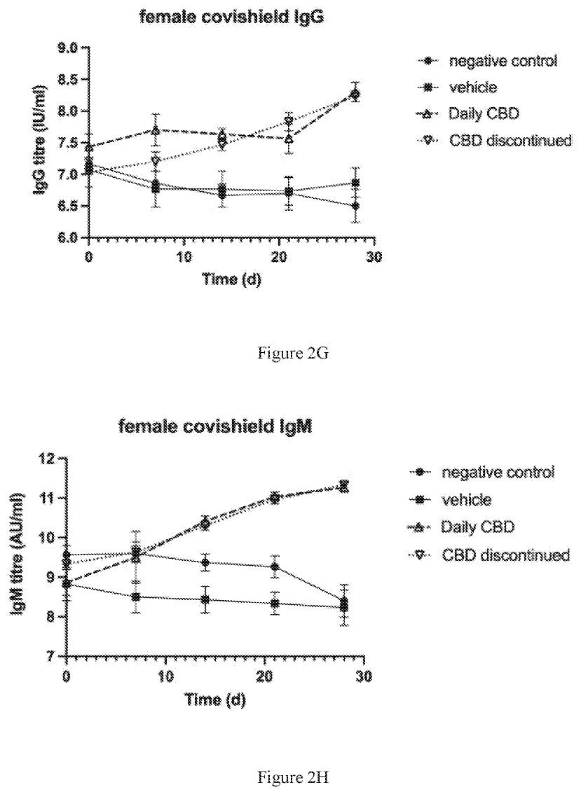

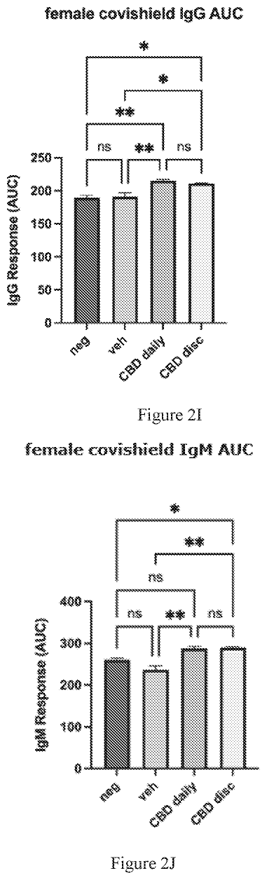

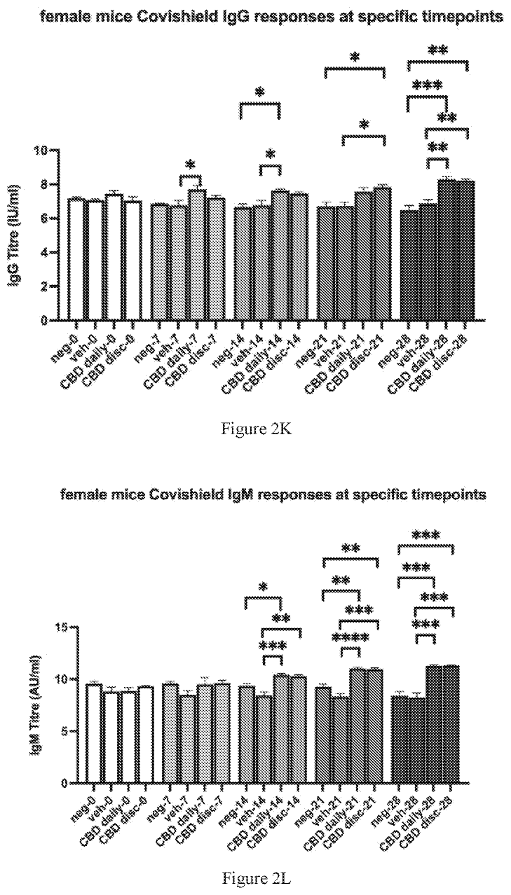

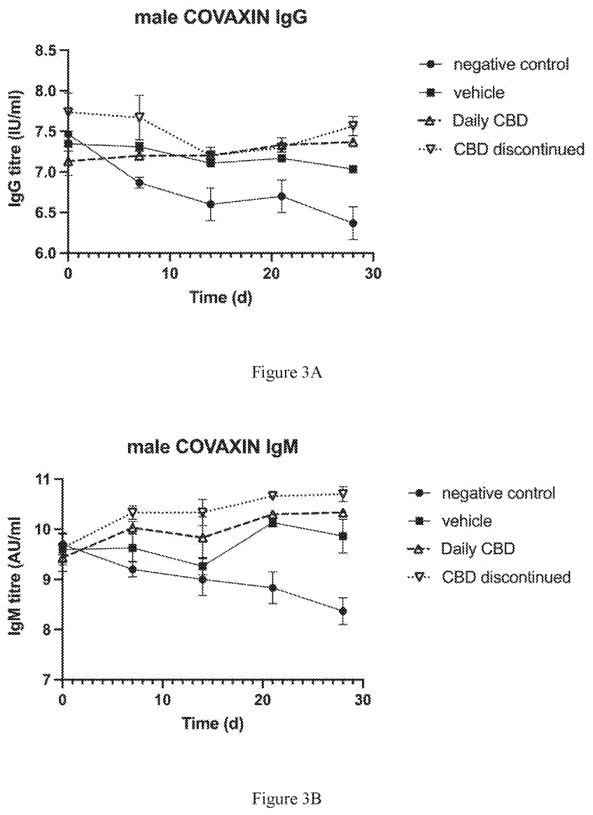

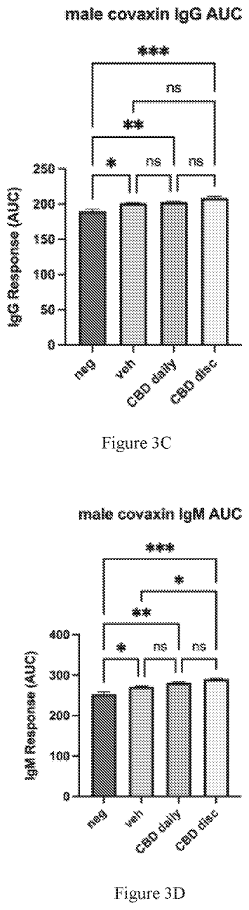

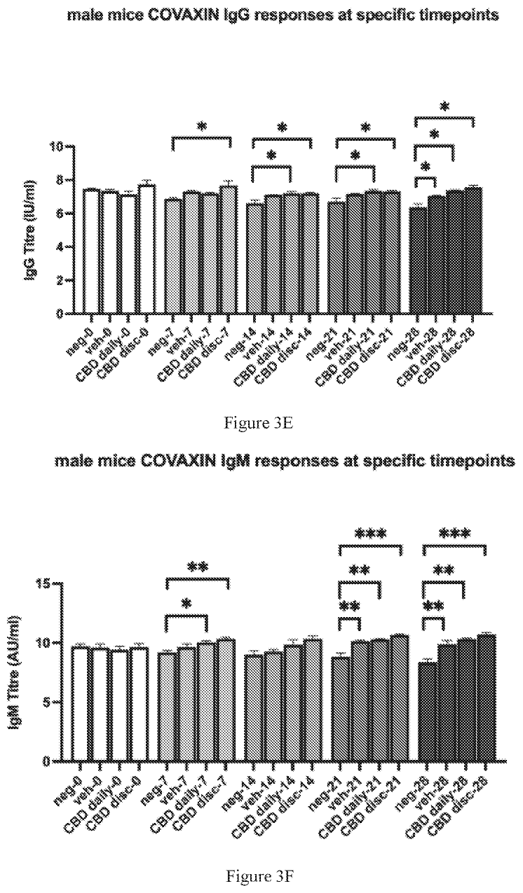

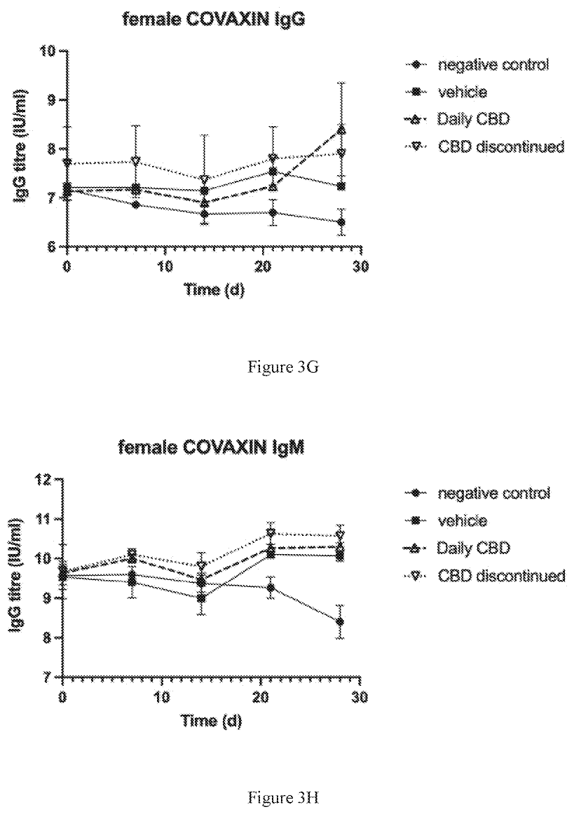

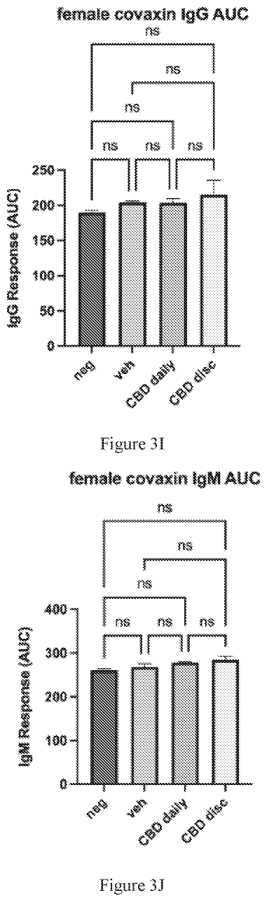

Effect of vaccines with and without Cannabidiol is reflected from FIGS. 2A-2L and FIGS. 3A-3J as described below in detail.

FIGS. 2A to 2L provide data for Covaxin.

FIGS. 2A-2L provide Antibody titre responses of male and female mice for Covishield vaccine. Four groups were studied in both male and female categories which are i) negative control (neg-no CBD, no vaccine), ii) vehicle control (veh—no CBD, vaccine at d0), iii) CBD daily (102.5 mg/kg/d from day −7 to day 28, with vaccine administered at d0), and iv) CBD discontinued after administration of vaccine (102.5 mg/kg/d CBD provided from day −7 to 0, with vaccine administered at d0). IgG and IgM titres were analyzed in blood collected humanely and non-lethally at days 0, 7, 14, 21, and 28. Data shown are means +/−SEM. *P<0.05, **P<0.01, ***P<0.001, ****P<0.0001. n=3.

FIGS. 2A and 2B respectively provide IgG (FIG. 2A) and IgM (FIG. 2B) antibody titres measured at various time points. Except for the initial couple of days, both IgG and IgM titer values are higher than negative control and vaccine alone (vehicle).

FIGS. 2C and 2D demonstrate that vaccine alone created a very poor IgG and IgM response that was not significantly different from the response seen in mice that were not vaccinated and the figures also provide that CBD significantly augmented the overall IgG and IgM responses to vaccine by 15% and 25%, respectively. The magnitude of the effect of CBD was the same if CBD was administered throughout the study, or just until the day that the vaccine was administered, suggesting that it acts to augment the efficacy of the vaccine at an early step after administration. Data were analyzed between by 1-way ANOVA followed by Tukey's post-hoc test for multiple comparisons among all groups.

FIGS. 2E and 2F-FIGS. 2E and 2F show that at any time point measured, mice injected with vaccine, but not given CBD, failed to have a significant antibody response compared to mice not given vaccine. However, in mice given CBD, either throughout the study, or just until administration of the vaccine, the IgG and IgM responses to the vaccine were significantly elevated already by 7 days after administration of the vaccine compared to mice not given CBD. Data were analyzed between by 1-way ANOVA followed by Tukey's post-hoc test for multiple comparisons among groups within each individual time point.

It is surprisingly found that antibody titres in mice given vaccine plus CBD rose and remained elevated until day 28, whereas antibody titres in mice injected with vaccine but not given CBD did not rise compared to mice not given vaccine. Thus CBD appears to play a significant role throughout the tenure tested and it further may continue to have such pronounced effects.

FIGS. 2G and 2H provide IgG (FIG. 2G) and IgM (FIG. 2H) antibody titres measured at various timepoints are shown. Except for the initial couple of days, both IgG and IgM titer values are higher than negative control and vaccine alone (vehicle).

FIGS. 2I and 2J provide area under the curves (AUC) for each mouse and graphed to show the average overall IgG response (FIG. 2I) and IgM response (FIG. 2J) over 28 days. The figures demonstrate that vaccine alone created a very poor IgG and IgM response that was not significantly different from the response seen in mice that were not vaccinated, but CBD significantly augmented the overall IgG and IgM responses to vaccine by 13% and 21%, respectively. The magnitude of the effect of CBD was the same if CBD was administered throughout the study, or just until the day that the vaccine was administered, suggesting that it acts to augment the efficacy of the vaccine at an early step after administration. Data were analyzed between by 1-way ANOVA followed by Tukey's post-hoc test for multiple comparisons among all groups.

FIGS. 2K and 2L provide that at any time point measured, mice injected with vaccine, but not given CBD, failed to have a significant antibody response compared to mice not given vaccine. However, in mice given CBD, either throughout the study, or just until administration of the vaccine, the IgG and IgM responses to the vaccine were significantly elevated already by 14 days after administration of the vaccine compared to mice not given CBD. In mice given CBD throughout the trial, there was a significantly better IgG response at day 7 than in the vehicle (no CBD) group, indicating that continuing CBD throughout the trial may help to augment the early IgG response in female mice. Data were analyzed between by 1-way ANOVA followed by Tukey's post-hoc test for multiple comparisons among groups within each individual timepoint.

Antibody titres in mice given vaccine plus CBD rose and remained elevated until day 28, whereas antibody titres in mice injected with vaccine but not given CBD did not rise compared to mice not given vaccine.

FIGS. 3A to 3J provide data for Covaxin.

FIGS. 3A and 3B provide that Vaccination of mice with Covaxin caused an increase in IgG and IgM responses, either when given alone or when given with CBD continuously, or CBD given prior to injection (and discontinued at injection), compared to mice that were not vaccinated. CBD given prior to injection augmented the overall IgM response to Covaxin versus Covaxin alone. An analysis of the response at timepoint 0, 7, 14, 21, and 28 days shows that in mice treated with CBD prior to injection (but then discontinued), there was an earlier response to Covaxin, such that a significant rise in IgG and IgM was apparent already at day 7. In mice treated throughout the study with CBD, there was also an earlier rise in IgG, already by day 14, and an earlier rise in IgM already at day 7. In contrast, a rise in IgG in mice vaccinated with Covaxin, but not given CBD, was not apparent until day 28, and a rise in IgM was not apparent until day 21.

An analysis of the response at timepoint 0, 7, 14, 21, and 28 days shows that in mice treated with CBD prior to injection (but then discontinued), there was an earlier response to Covaxin, such that a significant rise in IgG and IgM was apparent already at day 7. In mice treated throughout the study with CBD, there was also an earlier rise in IgG, already by day 14, and an earlier rise in IgM already at day 7. In contrast, a rise in IgG in mice vaccinated with Covaxin, but not given CBD, was not apparent until day 28, and a rise in IgM was not apparent until day 21.

The augmentation of the response to Covaxin by CBD tended to be less than the augmentation of the response to Covishield, but this was largely due to the greater response of mice to Covaxin, whereas there was no significant response by mice to Covishield.

RNA-type vaccines and DNA-type vaccines (such as COVISHIELD) deliver RNAs coding for one or more viral proteins (typically the SPIKE protein) into the muscle of a mammal. Cells in the muscle must make these proteins and then display them so that helper T-cells can recognize the foreign protein as a foreign antigen, and can process that antigen, and display it to B-cells that can make antibodies to that foreign antigen. Those antibodies provide defense against an initial infection, helping both to prevent infection, and to fight off an infection. In order for this to happen, immunosurveillance by circulating T-cells is necessary. In this regard, a therapy that can increase the innate immune response of muscle cells to the presence of viral RNA or viral DNA, such as by increasing the production of interferons by cells, can help to attract and recruit T-cells and other immunocytes to the area of inoculation. An enhanced T-cell presence at the site of inoculation would be expected to increase antigen uptake through phagocytosis, which is followed by T-cell presentation of antigen fragments to B-cells, resulting in an enhanced antibody generation by B-cells in response to the vaccine.

The finding that CBD can augment the antibody production following vaccination strongly suggests that CBD augments the antigenic response to vaccination with an RNA-type or DNA-type vaccine. It is expected that CBD would augment the antibody production following vaccination for any vaccine for Covid-19.

It is surprising that CBD has been reported in literature as an anti-inflammatory and anti-inflammatory role of CBD teaches away from the finding of the present study that the CBD augments antibody production following vaccination.

The present invention has surprisingly led to several outcomes.

Cannabidiol when administered with a Covid-19 vaccine to a mammal/human maintains or enhances response/level of IgG or IgM in such mammal/human for at least 14 days, preferably for at least 21 days, more preferably for at least 28 days.

Cannabidiol when administered with a Covid-19 vaccine to a mammal/human enhances response/level of IgG or IgM in such mammal/human wherein the composition enhances level of IgG or IgM within seven days or in less than 7 days.

Combination of Covid-19 vaccine of any type and Cannabidiol in suitable form such as pharmaceutically effective amount of Cannabidiol potentiate effect of vaccine by enhancing IgG as well as IgM levels/responses. The area under the curve indicates that the enhancement is at least 10% and as high as 25%. This effect of enhancement of at least 10% is seen for at least 14 days, preferably at least 21 days and more preferably at least 28 days. The end result which is a titer reading (IgG and IgM) on 28^{th} day is significantly higher and on a rising side/curve than the one without such combined administration which indicates that such high titer would continue longer.

Surprisingly even when CBD administration is stopped after vaccine administration, this rise is seen for further 28 days.

The present study thus can provide various options for combining a Covid-19 vaccine and Cannabidiol.

One more surprising effect was that the initial titer i.e. levels or response of IgG or IgM are quickly enhanced due to combined administration of vaccine and cannabidiol. Generally, a vaccine alone cannot achieve such levels so fast and take much longer time to achieve the same. With Cannabidiol composition, vaccines achieve significant rise in titer within seven days and even within a period shorter than 7 days. Cannabidiol enhances/boosts immune response administration of vaccine until development of immunity by vaccine. This is particularly useful for elderly population, immune-compromised population, population with comorbidities etc.

The invention thus proposes a kit comprising Cannabidiol and a Covid-19 vaccine where vaccine is selected from one or more of a component viral vaccine or one or more of a whole virus vaccine or any combination thereof.

Following tables provide IgG and IgM measured on 0, 7, 14, 21, and 28 days.

TABLE 1A
0 Day

| | | | Test | | |
|---|---|---|---|---|---|
| | | | Name | Units | Results |
| | | | PANEL EXTENDED, IgG & | | |
| | | | IgM, SERUM | | |
| Test Item | Group | | (CLIA, EIA) | | |
| Covaxin | Vehicle | GI a1F | IgG | IU/mL | 7.21 |
| | Formulation | | IgM | AU/mL | 9.8 |
| | | GI a2F | IgG | IU/mL | 7.19 |
| | | | IgM | AU/mL | 9.9 |
| | | GI a3F | IgG | IU/mL | 7.22 |
| | | | IgM | AU/mL | 8.9 |
| | | GI a4M | IgG | IU/mL | 7.22 |
| | | | IgM | AU/mL | 9.2 |
| | | GI a5M | IgG | IU/mL | 7.3 |
| | | | IgM | AU/mL | 10.2 |
| | | GI a6M | IgG | IU/mL | 7.52 |
| | | | IgM | AU/mL | 9.4 |
| | CBD | GII a1F | IgG | IU/mL | 7.4 |
| | Formulation | | IgM | AU/mL | 10.2 |
| | | GII a2F | IgG | IU/mL | 6.8 |
| | | | IgM | AU/mL | 9.2 |
| | | GII a3F | IgG | IU/mL | 7.2 |
| | | | IgM | AU/mL | 9.5 |
| | | GII a4M | IgG | IU/mL | 6.8 |
| | | | IgM | AU/mL | 9.6 |
| | | GII a5M | IgG | IU/mL | 7.2 |
| | | | IgM | AU/mL | 8.9 |
| | | GII a6M | IgG | IU/mL | 7.4 |
| | | | IgM | AU/mL | 9.8 |
| | CBD | GIII a1F | IgG | IU/mL | 7.1 |
| | Formulation | | IgM | AU/mL | 10.4 |
| | | GIII a2F | IgG | IU/mL | 9.2 |
| | | | IgM | AU/mL | 10.3 |
| | | GIII a3F | IgG | IU/mL | 6.8 |
| | | | IgM | AU/mL | 8.3 |
| | | GIII a4M | IgG | IU/mL | 7.5 |
| | | | IgM | AU/mL | 10.2 |
| | | GIII a5M | IgG | IU/mL | 8.21 |
| | | | IgM | AU/mL | 9.5 |
| | | GIII a6M | IgG | IU/mL | 7.5 |
| | | | IgM | AU/mL | 9.2 |

TABLE 1B
0 Day

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| | Control | GIVI a1F | IgG | IU/mL | 7.2 |
| | | | IgM | AU/mL | 9.8 |
| | | GIV a2F | IgG | IU/mL | 7.3 |
| | | | IgM | AU/mL | 9.1 |
| | | GIV a3F | IgG | IU/mL | 7 |
| | | | IgM | AU/mL | 9.8 |
| | | GIV a4M | IgG | IU/mL | 7.5 |
| | | | IgM | AU/mL | 10 |
| | | GIV a5M | IgG | IU/mL | 7.4 |
| | | | IgM | AU/mL | 9.3 |
| | | GIV a6M | IgG | IU/mL | 7.5 |
| | | | IgM | AU/mL | 9.8 |

TABLE 1C
0 Day

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| Covishild | Vehicle | GI (CS) | IgG | IU/mL | 7.21 |
| | Formulation | a1F | IgM | AU/mL | 938 |
| | | GI (CS) | IgG | IU/mL | 7 |
| | | a2F | IgM | AU/mL | 9.1 |
| | | GI (CS) | IgG | IU/mL | 7 |
| | | a3F | IgM | AU/mL | 8 |
| | | GI (CS) | IgG | IU/mL | 7.12 |
| | | a4M | IgM | AU/mL | 8.2 |
| | | GI (CS) | IgG | IU/mL | 7.1 |
| | | a5M | IgM | AU/mL | 9.1 |
| | | GI (CS) | IgG | IU/mL | 7.2 |
| | | a6M | IgM | AU/mL | 9.1 |
| | CBD | GII (CS) | IgG | IU/mL | 7.4 |
| | Formulation | a1F | IgM | AU/mL | 9.2 |
| | | GII (CS) | IgG | IU/mL | 7.8 |
| | | a2F | IgM | AU/mL | 8.2 |
| | | GII (CS) | IgG | IU/mL | 7.1 |
| | | a3F | IgM | AU/mL | 9.2 |
| | | GII (CS) | IgG | IU/mL | 6.8 |
| | | a4M | IgM | AU/mL | 10.1 |
| | | GII (CS) | IgG | IU/mL | 7.1 |
| | | a5M | IgM | AU/mL | 9.4 |
| | | GII (CS) | IgG | IU/mL | 6.4 |
| | | a6M | IgM | AU/mL | 8.8 |
| | CBD | GIII (CS) | IgG | IU/mL | 7.5 |
| | Formulation | a1F | IgM | AU/mL | 9.4 |
| | | GIII (CS) | IgG | IU/mL | 6.8 |
| | | a2F | IgM | AU/mL | 9.3 |
| | | GIII (CS) | IgG | IU/mL | 6.8 |
| | | a3F | IgM | AU/mL | 9.3 |
| | | GIII (CS) | IgG | IU/mL | 6.5 |
| | | a4M | IgM | AU/mL | 9.2 |
| | | GIII (CS) | IgG | IU/mL | 7.21 |
| | | a5M | IgM | AU/mL | 9.3 |
| | | GIII (CS) | IgC | IU/mL | 7.2 |
| | | a6M | IgM | AU/mL | 9.1 |

TABLE 2A
7th Day

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| Covaxin | Vehicle | GI b1F | IgG | IU/mL | 7.22 |
| | Formulation | | IgM | AU/mL | 9.8 |
| | | GI b2F | IgG | IU/mL | 7.19 |
| | | | IgM | AU/mL | 9.8 |
| | | GI b3F | IgG | IU/mL | 7.21 |
| | | | IgM | AU/mL | 8.6 |
| | | GI b4M | IgG | IU/mL | 7.31 |
| | | | IgM | AU/mL | 9.8 |
| | | GI b5M | IgG | IU/mL | 7.4 |
| | | | IgM | AU/mL | 10 |
| | | GI b6M | IgG | IU/mL | 7.22 |
| | | | IgM | AU/mL | 9.1 |
| | CBD | GII b1F | IgG | IU/mL | 7.2 |
| | Formulation | | IgM | AU/mL | 10.2 |
| | | GII b2F | IgG | IU/mL | 7.1 |
| | | | IgM | AU/mL | 9.8 |
| | | GII b3F | IgG | IU/mL | 7.2 |
| | | | IgM | AU/mL | 10 |
| | | GII b4M | IgG | IU/mL | 7.1 |
| | | | IgM | AU/mL | 10.2 |
| | | GII b5M | IgG | IU/mL | 7.3 |
| | | | IgM | AU/mL | 10.1 |
| | | GII b6M | IgG | IU/mL | 7.2 |
| | | | IgM | AU/mL | 9.8 |
| | CBD | GIII b1F | IgG | IU/mL | 6.9 |
| | Formulation | | IgM | AU/mL | 10.2 |
| | | GIII b2F | IgG | IU/mL | 9.2 |
| | | | IgM | AU/mL | 10.3 |
| | | GIII b3F | IgG | IU/mL | 7.1 |
| | | | IgM | AU/mL | 9.8 |
| | | GIII b4M | IgG | IU/mL | 7.5 |
| | | | IgM | AU/mL | 10.2 |
| | | GIII b5M | IgG | IU/mL | 8.21 |
| | | | IgM | AU/mL | 10.6 |
| | | GIII b6M | IgG | IU/mL | 7.3 |
| | | | IgM | AU/mL | 10.2 |

TABLE 2B
7th Day

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| | Control | GIVI b1F | IgG | IU/mL | 6.8 |
| | | | IgM | AU/mL | 9.8 |
| | | GIV b2F | IgG | IU/mL | 6.9 |
| | | | IgM | AU/mL | 9.2 |
| | | GIV b3F | IgG | IU/mL | 6.88 |
| | | | IgM | AU/mL | 9.8 |
| | | GIV b4M | IgG | IU/mL | 7 |
| | | | IgM | AU/mL | 8.9 |
| | | GIV b5M | IgG | IU/mL | 6.8 |
| | | | IgM | AU/mL | 9.3 |
| | | GIV b6M | IgG | IU/mL | 6.8 |
| | | | IgM | AU/mL | 9.4 |

TABLE 2C
7th Day

| | | | Test | | |
|---|---|---|---|---|---|
| | | | Name | Units | Results |
| | | | PANEL EXTENDED, IgG & | | |
| Test | | | IgM, SERUM | | |
| Item | Group | | (CLIA, EIA) | | |
| Covishild | Vehicle | GI (CS) | IgG | IU/mL | 7.1 |
| | Formulation | b1F | IgM | AU/mL | 9.3 |
| | | GI (CS) | IgG | IU/mL | 7 |
| | | b2F | IgM | AU/mL | 8.2 |
| | | GI (CS) | IgG | IU/mL | 6.2 |
| | | b3F | IgM | AU/mL | 8 |
| | | GI (CS) | IgG | IU/mL | 6.1 |
| | | b4M | IgM | AU/mL | 8.2 |
| | | GI (CS) | IgG | IU/mL | 6.8 |
| | | b5M | IgM | AU/mL | 9.2 |
| | | GI (CS) | IgG | IU/mL | 6.6 |
| | | b6M | IgM | AU/mL | 8.3 |
| | CBD | GII (CS) | IgG | IU/mL | 7.4 |
| | Formulation | b1F | IgM | AU/mL | 10.2 |
| | | GII (CS) | IgG | IU/mL | 7.5 |
| | | b2F | IgM | AU/mL | 8.2 |
| | | GII (CS) | IgG | IU/mL | 8.2 |
| | | b3F | IgM | AU/mL | 10.11 |
| | | GII (CS) | IgG | IU/mL | 7.3 |
| | | b4M | IgM | AU/mL | 10.8 |
| | | GII (CS) | IgG | IU/mL | 7.3 |
| | | b5M | IgM | AU/mL | 10.5 |
| | | GII (CS) | IgG | IU/mL | 7.8 |
| | | b6M | IgM | AU/mL | 10.4 |
| | CBD | GIII (CS) | IgG | IU/mL | 7.4 |
| | Formulation | b1F | IgM | AU/mL | 9.2 |
| | | GIII (CS) | IgG | IU/mL | 6.9 |
| | | b2F | IgM | AU/mL | 9.6 |
| | | GIII (CS) | IgG | IU/mL | 7.3 |
| | | b3F | IgM | AU/mL | 10.1 |
| | | GIII (CS) | IgG | IU/mL | 7.2 |
| | | b4M | IgM | AU/mL | 10.2 |
| | | GIII (CS) | IgG | IU/mL | 7.3 |
| | | b5M | IgM | AU/mL | 10.3 |
| | | GIII (CS) | IgG | IU/mL | 7.1 |
| | | b6M | IgM | AU/mL | 10.6 |

TABLE 3A
14 Day

| | | | Test | | |
|---|---|---|---|---|---|
| | | | Name | Units | Results |
| | | | PANEL EXTENDED, IgG & | | |
| Test | | | IgM, SERUM | | |
| Item | Group | | (CLIA, EIA) | | |
| Covaxin | Vehicle | GI c1F | IgG | IU/mL | 7.12 |
| | Formulation | | IgM | AU/mL | 9.6 |
| | | GI c2F | IgG | IU/mL | 7.1 |
| | | | IgM | AU/mL | 9.2 |
| | | GI c3F | IgG | IU/mL | 7.22 |
| | | | IgM | AU/mL | 8.2 |
| | | GI c4M | IgG | IU/mL | 7.1 |
| | | | IgM | AU/mL | 9.6 |
| | | GI c5M | IgG | IU/mL | 7.1 |
| | | | IgM | AU/mL | 9.2 |
| | | GI c6M | IgG | IU/mL | 7.12 |
| | | | IgM | AU/mL | 9 |
| | Daily CBD | GII c1F | IgG | IU/mL | 7.2 |
| | Formulation | | IgM | AU/mL | 9.3 |
| | | GII c2F | IgG | IU/mL | 6.4 |
| | | | IgM | AU/mL | 9.3 |
| | | GII c3F | IgG | IU/mL | 7.1 |
| | | | IgM | AU/mL | 9.8 |
| | | GII c4M | IgG | IU/mL | 7.1 |
| | | | IgM | AU/mL | 10.2 |
| | | GII c5M | IgG | IU/mL | 7.4 |
| | | | IgM | AU/mL | 10.3 |
| | | GII c6M | IgG | IU/mL | 7.1 |
| | | | IgM | AU/mL | 9 |
| | CBD | GIII c1F | IgG | IU/mL | 6 |
| | Formulation | | IgM | AU/mL | 10.4 |
| | | GIII c2F | IgG | IU/mL | 9.1 |
| | | | IgM | AU/mL | 9.8 |
| | | GIII c3F | IgG | IU/mL | 7 |
| | | | IgM | AU/mL | 9.2 |
| | | GIII | IgG | IU/mL | 7.2 |
| | | c4M | IgM | AU/mL | 9.8 |
| | | GIII | IgG | IU/mL | 7.3 |
| | | c5M | IgM | AU/mL | 10.6 |
| | | GIII | IgG | IU/mL | 7.1 |
| | | c6M | IgM | AU/mL | 10.6 |

TABLE 3B
14 Day

| | | | Test Name | Units | Results |
|---|---|---|---|---|---|
| Test | | | PANEL EXTENDED, IgG & IgM, SERUM | | |
| Item | Group | | (CLIA, EIA) | | |
| | Control | GIVI | IgG | IU/mL | 6.8 |
| | | c1F | IgM | AU/mL | 9.8 |
| | | GIV | IgG | IU/mL | 6.9 |
| | | c2F | IgM | AU/mL | 9.2 |
| | | GIV | IgG | IU/mL | 6.3 |
| | | c3F | IgM | AU/mL | 9.1 |
| | | GIV | IgG | IU/mL | 6.2 |
| | | c4M | IgM | AU/mL | 8.4 |
| | | GIV | IgG | IU/mL | 6.8 |
| | | c5M | IgM | AU/mL | 9.1 |
| | | GIV | IgG | IU/mL | 6.8 |
| | | c6M | IgM | AU/mL | 9.5 |

TABLE 3C
14 Day

| | | | Test | | |
|---|---|---|---|---|---|
| | | | Name | Units | Results |
| | | | PANEL EXTENDED, IgG & | | |
| Test | | | IgM, SERUM | | |
| Item | Group | | (CLIA, EIA) | | |
| Covishild | Vehicle | GI (CS) | IgG | IU/mL | 7.1 |
| | Formulation | c1F | IgM | AU/mL | 9.1 |
| | | GI (CS) | IgG | IU/mL | 7 |
| | | c2F | IgM | AU/mL | 8.2 |
| | | GI (CS) | IgG | IU/mL | 6.2 |
| | | c3F | IgM | AU/mL | 8 |
| | | GI (CS) | IgG | IU/mL | 6.1 |
| | | c4M | IgM | AU/mL | 8.2 |
| | | GI (CS) | IgG | IU/mL | 6.8 |
| | | c5M | IgM | AU/mL | 9.2 |
| | | GI (CS) | IgG | IU/mL | 6.6 |
| | | c6M | IgM | AU/mL | 8.3 |
| | CBD | GII (CS) | IgG | IU/mL | 7.5 |
| | Formulation | c1F | IgM | AU/mL | 10.6 |
| | | GII (CS) | IgG | IU/mL | 7.8 |
| | | c2F | IgM | AU/mL | 10.5 |
| | | GII (CS) | IgG | IU/mL | 7.6 |
| | | c3F | IgM | AU/mL | 10.15 |
| | | GII (CS) | IgG | IU/mL | 7.8 |
| | | c4M | IgM | AU/mL | 11 |
| | | GII (CS) | IgG | IU/mL | 7.8 |
| | | c5M | IgM | AU/mL | 10.52 |
| | | GII (CS) | IgG | IU/mL | 8.2 |
| | | c6M | IgM | AU/mL | 11 |
| | CBD | GIII (CS) | IgG | IU/mL | 7.5 |
| | Formulation | c1F | IgM | AU/mL | 10.1 |
| | | GIII (CS) | IgG | IU/mL | 7.6 |
| | | c2F | IgM | AU/mL | 10.5 |
| | | GIII (CS) | IgG | IU/mL | 7.3 |
| | | c3F | IgM | AU/mL | 10.3 |
| | | GIII (CS) | IgG | IU/mL | 7.5 |
| | | c4M | IgM | AU/mL | 10.3 |
| | | GIII (CS) | IgG | IU/mL | 7.8 |
| | | c5M | IgM | AU/mL | 11.2 |
| | | GIII (CS) | IgG | IU/mL | 7.9 |
| | | c6M | IgM | AU/mL | 11 |

TABLE 4A
21 Day

| | | | Test | | |
|---|---|---|---|---|---|
| | | | Name | Units | Results |
| Test | | | PANEL EXTENDED, IgG & IgM, SERUM | | |
| Item | Group | | (CLIA, EIA) | | |
| Covaxin | Vehicle | GI | IgG | IU/mL | 7.22 |
| | Formu- | d1F | IgM | AU/mL | 10.1 |
| | lation | GI | IgG | IU/mL | 7.3 |
| | | d2F | IgM | AU/mL | 10.2 |
| | | GI | IgG | IU/mL | 8.1 |
| | | d3F | IgM | AU/mL | 10 |
| | | GI | IgC | IU/mL | 7.1 |
| | | d4M | IgM | AU/mL | 10.1 |
| | | GI | IgG | IU/mL | 7.2 |
| | | d5M | IgM | AU/mL | 10 |
| | | GI | IgG | IU/mL | 7.2 |
| | | d6M | IgM | AU/mL | 10.3 |
| | CBD | GII | IgG | IU/mL | 7.3 |
| | Formu- | d1F | IgM | AU/mL | 10.2 |
| | lation | GII | IgG | IU/mL | 7.1 |
| | | d2F | IgM | AU/mL | 10.2 |
| | | GII | IgG | IU/mL | 7.3 |
| | | d3F | IgM | AU/mL | 10.4 |
| | | GII | IgG | IU/mL | 7.5 |
| | | d4M | IgM | AU/mL | 10.4 |
| | | GII | IgG | IU/mL | 7.2 |
| | | d5M | IgM | AU/mL | 10.3 |
| | | GII | IgG | IU/mL | 7.3 |
| | | d6M | IgM | AU/mL | 10.2 |
| | CBD | GIII | IgG | IU/mL | 7.3 |
| | Formu- | d1F | IgM | AU/mL | 11 |
| | lation | GIII | IgG | IU/mL | 9.1 |
| | | d2F | IgM | AU/mL | 10.8 |
| | | GIII | IgG | IU/mL | 7 |
| | | d3F | IgM | AU/mL | 10.1 |
| | | GIII | IgG | IU/mL | 7.4 |
| | | d4M | IgM | AU/mL | 10.6 |
| | | GIII | IgG | IU/mL | 7.2 |
| | | d5M | IgM | AU/mL | 10.6 |
| | | GIII | IgG | IU/mL | 7.3 |
| | | d6M | IgM | AU/mL | 10.8 |

TABLE 4B
21 Day

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| | Control | GIVI d1F | IgG | IU/mL | 7.1 |
| | | | IgM | AU/mL | 9.8 |
| | | GIV d2F | IgG | IU/mL | 6.8 |
| | | | IgM | AU/mL | 9.1 |
| | | GIV d3F | IgG | IU/mL | 6.2 |
| | | | IgM | AU/mL | 8.9 |
| | | GIV d4M | IgG | IU/mL | 6.3 |
| | | | IgM | AU/mL | 8.3 |
| | | GIV d5M | IgG | IU/mL | 6.9 |
| | | | IgM | AU/mL | 8.8 |
| | | GIV d6M | IgG | IU/mL | 6.9 |
| | | | IgM | AU/mL | 9.4 |

TABLE 4C
21 Day

| | | | Test | | |
|---|---|---|---|---|---|
| | | | Name | Units | Results |
| | | | PANEL EXTENDED, IgG & IgM, | | |
| Test | | | SERUM | | |
| Item | Group | | (CLIA, EIA) | | |
| Covishild | Vehicle | GI (CS) d1F | IgG | IU/mL | 7 |
| | Formu- | | IgM | AU/mL | 8.9 |
| | lation | GI (CS) d2F | IgG | IU/mL | 6.9 |
| | | | IgM | AU/mL | 8 |
| | | GI (CS) d3F | IgG | IU/mL | 6.3 |
| | | | IgM | AU/mL | 8.1 |
| | | GI (CS) | IgG | IU/mL | 7.1 |
| | | d4M | IgM | AU/mL | 8.2 |
| | | GI (CS) | IgG | IU/mL | 6.4 |
| | | d5M | IgM | AU/mL | 8.9 |
| | | GI (CS) | IgG | IU/mL | 6.8 |
| | | d6M | IgM | AU/mL | 8.6 |
| | CBD | GII (CS) | IgG | IU/mL | 7.8 |
| | Formu- | d1F | IgM | AU/mL | 10.8 |
| | lation | GII (CS) | IgG | IU/mL | 7.1 |
| | | d2F | IgM | AU/mL | 11.1 |
| | | GII (CS) | IgG | IU/mL | 7.8 |
| | | d3F | IgM | AU/mL | 11.2 |
| | | GII (CS) | IgG | IU/mL | 7.5 |
| | | d4M | IgM | AU/mL | 11 |
| | | GII (CS) | IgG | IU/mL | 7.9 |
| | | d5M | IgM | AU/mL | 11 |
| | | GII (CS) | IgG | IU/mL | 8.1 |
| | | d6M | IgM | AU/mL | 11.12 |
| | CBD | GIII (CS) | IgG | IU/mL | 7.6 |
| | Formu- | d1F | IgM | AU/mL | 10.8 |
| | lation | GIII (CS) | IgG | IU/mL | 8.1 |
| | | d2F | IgM | AU/mL | 10.9 |
| | | GIII (CS) | IgG | IU/mL | 7.8 |
| | | d3F | IgM | AU/mL | 11.2 |
| | | GIII (CS) | IgG | IU/mL | 8.1 |
| | | d4M | IgM | AU/mL | 11.2 |
| | | GIII (CS) | IgG | IU/mL | 8.1 |
| | | d5M | IgM | AU/mL | 11.2 |
| | | GIII (CS) | IgG | IU/mL | 7.6 |
| | | d6M | IgM | AU/mL | 11.2 |

TABLE 5A
28 Days

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| Covaxin | Vehicle | GI e1F | IgG | IU/mL | 7.3 |
| | Formulation | | IgM | AU/mL | 10.2 |
| | | GI e2F | IgG | IU/mL | 7.1 |
| | | | IgM | AU/mL | 10.2 |
| | | GI e3F | IgG | IU/mL | 7.3 |
| | | | IgM | AU/mL | 9.8 |
| | | GI e4M | IgG | IU/mL | 7 |
| | | | IgM | AU/mL | 10.2 |
| | | GI e5M | IgG | IU/mL | 7.1 |
| | | | IgM | AU/mL | 9.2 |
| | | GI e6M | IgG | IU/mL | 7 |
| | | | IgM | AU/mL | 10.2 |
| | CBD | GII e1F | IgG | IU/mL | 10.3 |
| | Formulation | | IgM | AU/mL | 10.2 |
| | | GII e2F | IgG | IU/mL | 7.5 |
| | | | IgM | AU/mL | 10.2 |
| | | GII e3F | IgG | IU/mL | 7.4 |
| | | | IgM | AU/mL | 10.5 |
| | | GII e4M | IgG | IU/mL | 7.4 |
| | | | IgM | AU/mL | 10.4 |
| | | GII e5M | IgG | IU/mL | 7.3 |
| | | | IgM | AU/mL | 10.2 |
| | | GII e6M | IgG | IU/mL | 7.4 |
| | | | IgM | AU/mL | 10.4 |
| | CBD | GIII e1F | IgG | IU/mL | 7.4 |
| | Formulation | | IgM | AU/mL | 11.1 |
| | | GIII e2F | IgG | IU/mL | 9.1 |
| | | | IgM | AU/mL | 10.4 |
| | | GIII e3F | IgG | IU/mL | 7.2 |
| | | | IgM | AU/mL | 10.2 |
| | | GIII e4M | IgG | IU/mL | 7.8 |
| | | | IgM | AU/mL | 10.6 |
| | | GIII e5M | IgG | IU/mL | 7.4 |
| | | | IgM | AU/mL | 10.5 |
| | | GIII e6M | IgG | IU/mL | 7.5 |
| | | | IgM | AU/mL | 11 |

TABLE 5B
28 Days

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| | Control | GIVI e1F | IgG | IU/mL | 7 |
| | | | IgM | AU/mL | 8.2 |
| | | GIV e2F | IgG | IU/mL | 6.1 |
| | | | IgM | AU/mL | 9.2 |
| | | GIV e3F | IgG | IU/mL | 6.4 |
| | | | IgM | AU/mL | 7.8 |
| | | GIV e4M | IgG | IU/mL | 6 |
| | | | IgM | AU/mL | 8.1 |
| | | GIV e5M | IgG | IU/mL | 6.7 |
| | | | IgM | AU/mL | 8.1 |
| | | GIV e6M | IgG | IU/mL | 6.4 |
| | | | IgM | AU/mL | 8.9 |

TABLE 5C
28 Days

| | | | Test Name | Units | Results |
|---|---|---|---|---|---|
| | | | PANEL EXTENDED, IgG & IgM, | | |
| Test | | | SERUM | | |
| Item | Group | | (CLIA, EIA) | | |
| Covishild | Vehicle | GI (CS) e1F | IgG | IU/mL | 7.1 |
| | Formu- | | IgM | AU/mL | 9.1 |
| | lation | GI (CS) e2F | IgG | IU/mL | 7.1 |
| | | | IgM | AU/mL | 7.6 |
| | | GI (CS) e3F | IgG | IU/mL | 6.4 |
| | | | IgM | AU/mL | 8 |
| | | GI (CS) e4M | IgG | IU/mL | 7.2 |
| | | | IgM | AU/mL | 8.1 |
| | | GI (CS) e5M | IgG | IU/mL | 6.2 |
| | | | IgM | AU/mL | 8.2 |
| | | GI (CS) e6M | IgG | IU/mL | 6.5 |
| | | | IgM | AU/mL | 8.3 |
| | CBD | GII (CS) e1F | IgG | IU/mL | 8.6 |
| | Formu- | | IgM | AU/mL | 11.1 |
| | lation | GII (CS) e2F | IgG | IU/mL | 8.1 |
| | | | IgM | AU/mL | 11.2 |
| | | GII (CS) e3F | IgG | IU/mL | 8.2 |
| | | | IgM | AU/mL | 11.5 |
| | | GII (CS) | IgG | IU/mL | 7.8 |
| | | e4M | IgM | AU/mL | 11.6 |
| | | GII (CS) | IgG | IU/mL | 8.2 |
| | | e5M | IgM | AU/mL | 11.4 |
| | | GII (CS) | IgG | IU/mL | 8.4 |
| | | e6M | IgM | AU/mL | 11.2 |
| | CBD | GIII (CS) | IgG | IU/mL | 8.1 |
| | Formu- | e1F | IgM | AU/mL | 11.3 |
| | lation | GIII (CS) | IgG | IU/mL | 8.4 |
| | | e2F | IgM | AU/mL | 11.3 |
| | | GIII (CS) | IgG | IU/mL | 8.2 |
| | | e3F | IgM | AU/mL | 11.4 |
| | | GIII (CS) | IgG | IU/mL | 8.3 |
| | | e4M | IgM | AU/mL | 11.5 |
| | | GIII (CS) | IgG | IU/mL | 8.4 |
| | | e5M | IgM | AU/mL | 11.8 |
| | | GIII (CS) | IgG | IU/mL | 8.2 |
| | | e6M | IgM | AU/mL | 11.6 |

Following examples describe invention without limiting the scope of the invention in any way

### EXAMPLE 1

### General Protocol And Conditions

a). Conditions: The animals are housed under standard laboratory conditions, airconditioned with adequate fresh air supply (Air changes 12-16 per hour), room temperature of 22±3C, relative humidity at 30-70%, with 12 hours light and 12 hours dark cycle. The temperature and relative humidity are recorded daily.

b). Housing: Five animals are housed per cage in standard polypropylene cages having the size: Length 43.0×Breadth 27.0×Height 15.0 cm with top stainless steel grill mesh with top grill having facilities for holding pellet feed and drinking water in water bottle fitted with stainless steel sipper tube.

c). Acclimatization: The animals are acclimatized for a period of five days before the start of the study to allow the animals for acclimatization to laboratory conditions and are observed for clinical signs daily. Veterinary examination of all the animals are recorded on the day of receipt and on the day of randomization for grouping.

d). Diet: The animals are fed ad libitum throughout the acclimatization and study period. Rodent feed manufactured by VRK Scientists Choice and procured from Champaka Feed and Foods, Bengaluru, India was provided.

e) Water: Clean uncontaminated drinking water is provided ad libitum throughout the acclimatization and study period. Deep bore-well water passed through reverse osmosis and exposed to UV rays in Aqua guard water filter cum purifier manufactured by Eureka Forbes Ltd., Mumbai, India is provided in plastic water bottles with stainless steel sipper tubes.

f). Bedding Material: Clean and sieved paddy husk is used as bedding material for the present study.

g) Test Items: The test items formulations was prepared afresh before dosing. The test items

provided by the sponsor were weighed and the doses calculated based on animal's body

weight. All the doses of the test items are administered at the same volume 0.1-0.2 ml of prepared test items as per standard approved guidelines. The vaccine was administered intramuscularly.

i) Blood samples. Blood samples were collected from the animals from the orbital venous plexus in 1.5 ml microfuge tubes without any anticoagulant. The blood samples were centrifuged at 3000 rpm for 10 minutes to obtain the serum samples within 1-2 hours after the collection. The serum samples were analyzed for the antibody titers (IgG and IgM).

### EXAMPLE 2

### Mice Were Divided Into Four Groups as Follows

1. Negative control—This group was not given a vaccine, and was not administered CBD.

2. Vehicle control—This group was given vaccine given at time point 0, then gavaged with carrier oil only (no CBD)).

3. CBD daily for 7 days before vaccine and continued after administration of vaccine for 28 days. Mice were gavaged daily with 102.5 mg/kg Cannabidiol (CBD) for 7 days (starting at timepoint=−7 days) then given vaccine at day 0, then gavaged daily for the following 28 days with 102.5 mg/kg/day CBD). Thus, Cannabidiol is administered for a total of 35 days.

4. CBD daily for 7 days before vaccine and discontinued after administration of vaccine-Mice were gavaged daily with 102.5 mg/kg CBD for 7 days (starting at time point=−7 days) then given vaccine at day 0. CBD discontinued after vaccination at day 0.

CBD formulations employed in example 2 contained solution of CBD in carrier oil which is a sesame oil.

Blood samples were taken at day 0 (i.e. on the day of vaccine administration), and on 7, 14, 21, and 28 from the orbital venous plexus for analysis of IgG and IgM levels. The IgG and IgM levels found on day 0 are reported in tables 1A to 1C. The IgG and IgM levels found on day 7 are reported in tables 2A to 2C. The IgG and IgM levels found on day 21 are reported in tables 3A to 3C. The IgG and IgM levels found on day 28 are reported in tables 4A to 4C. Effect of vaccines with and without Cannabidiol is reflected from FIGS. 2A-2L and FIGS. 3A-3J.

### REFERENCES

Thomas S J, Moreira E D Jr, Kitchin N, Absalon J, Gurtman A, Lockhart S, Perez J L, Pérez Marc G, Polack F P, Zerbini C, Bailey R, Swanson K A, Xu X, Roychoudhury S, Koury K, Bouguermouh S, Kalina W V, Cooper D, Frenck R W Jr, Hammitt L L, Türeci O, Nell H, Schaefer A, Unal S, Yang Q, Liberator P, Tresnan D B, Mather S, Dormitzer P R, Şahin U, Gruber W C, Jansen K U; C4591001 Clinical Trial Group. Safety and Efficacy of the BNT162b2 mRNA Covid-19 Vaccine through 6 Months. N Engl J Med. 2021 Nov. 4; 385(19): 1761-1773. doi: 10.1056/NEJMoa2110345. Epub 2021 Sep. 15. PMID: 34525277; PMCID: PMC8461570.

Fouda M A, Ghovanloo M R, Ruben P C. Cannabidiol protects against high glucose-induced oxidative stress and cytotoxicity in cardiac voltage-gated sodium 5 channels. Br J Pharmacol. 2020 Feb. 19.

Kenneth E. Remy, Monty Mazer, David A. Striker, Ali H. Ellebedy, Andrew H. Walton, Jacqueline Unsinger, Teresa M. Blood, Philip A. Mudd, Daehan J. Yi, Daniel A. Mannion, Dale F. Osborne, R. Scott Martin, Nitin J. Anand, James P. Bosanquet, Jane Blood, Anne M. Drewry, Charles C. Caldwell, Isaiah R. Turnbull, Scott C. Brakenridge, Lyle L. Moldwawer, Richard S. Hotchkiss., Severe immunosuppression and not a cytokine storm characterizes COVID-19 infections, JCI Insight. 2020; 5(17): e140329. https://doi.org/10.1172/cL.insight.140329.

Cyranoski D. Profile of a killer: the complex biology powering the coronavirus pandemic. Nature. 2020 May;581 (7806): 22-26. doi: 10.1038/d41586-020-01315-7. PMID: 32367025.

Jeyanathan, M., Afkhami, S., Smaill, F. et al. Immunological considerations for COVID-19 vaccine strategies. Nat Rev Immunol 20, 615-632 (2020). https://doi.org/10.1038/s41577-020-00434-6

Lurie N, Saville M, Hatchett R, Halton J. Developing Covid-19 Vaccines at Pandemic Speed. N Engl J Med. 2020 May 21;382 (21): 1969-1973. doi: 10.1056/NEJMp2005630. Epub 2020 Mar. 30. PMID: 32227757.

Blanco-Melo D, Nilsson-Payant B E, Liu W C, Uhl S, Hoagland D, Møller R, Jordan T X, Oishi K, Panis M, Sachs D, Wang T T, Schwartz R E, Lim J K, Albrecht R A, tenOever B R. Imbalanced Host Response to SARS-COV-2 Drives Development of COVID-19. Cell. 2020 May 28;181 (5): 1036-1045.e9. doi: 10.1016/j.cell.2020.04.026. Epub 2020 May 15. PMID: 32416070; PMCID: PMC7227586.

1. A pharmaceutical composition comprising therapeutically effective amount of Cannabidiol for administration with a Covid-19 vaccine to a mammal/human to sustain and/or enhance effect of vaccine.

2. The pharmaceutical composition according to the claim 1 when administered with a Covid-19 vaccine to a mammal/human maintains or enhances response/level of one or more antibodies selected from IgG and IgM.

3. The pharmaceutical composition according to the claim 2 when administered with a Covid-19 vaccine to a mammal/human maintains or enhances response/level of IgG or IgM in such mammal/human for at least 14 days.

4. The pharmaceutical composition according to the claim 2 when administered with a Covid-19 vaccine to a mammal/human maintains or enhances response/level of IgG or IgM in such mammal/human for at least 21 days.

5. The pharmaceutical composition according to the claim 2 when administered with a Covid-19 vaccine to a mammal/human maintains or enhances response/level of IgG or IgM in such mammal/human for at least 28 days.

6. The pharmaceutical composition according to the claim 2 when administered with a Covid-19 vaccine to a mammal/human enhances response/level of IgG or IgM in such mammal/human wherein the composition enhances level of IgG or IgM within seven days.

7. The pharmaceutical composition according to the claim 2 when administered with a Covid-19 vaccine to a mammal/human enhances response/level of IgG or IgM in such mammal/human by at least 10%.

8. The pharmaceutical composition according to the claim 7 when administered with a Covid-19 vaccine to a mammal/human maintains or enhances response/level of IgG or IgM in such mammal/human by at least 10% for at least 14 days.

9. The pharmaceutical composition according to the claim 7 when administered with a Covid-19 vaccine to a mammal/human maintains or enhances response/level of IgG or IgM in such mammal/human by at least 10% for at least 21 days.

10. The pharmaceutical composition according to the claim 7 when administered with a Covid-19 vaccine to a mammal/human maintains or enhances response/level of IgG or IgM in such mammal/human by at least 10% for at least 28 days.

11. A method to sustain and/or enhance effect of a Covid-19 vaccine in a mammal/human comprising administering to such a mammal/human a pharmaceutical composition comprising a therapeutically effective amount of Cannabidiol along with a Covid-19 vaccine.

12. The method of administering the pharmaceutical composition and a Covid 19 vaccine according to the claim 11 in a mammal/human wherein sustenance or enhancement of effect of Covid-19 vaccine comprises maintenance or enhancement of response/level of one or more antibodies selected from IgG and IgM.

13. The method according to the claim 12 wherein maintenance or enhancement of response/level of IgG or IgM is for at least 14 days.

14. The method according to the claim 12 wherein maintenance or enhancement of response/level of IgG or IgM is for at least 21days.

15. The method according to the claim 12 wherein maintenance or enhancement of response/level of IgG or IgM is for at least 28 days.

16. The method according to the claim 12 wherein response/level of IgG or IgM is enhanced within seven days.

17. The method according to the claim 12 wherein response/level of IgG or IgM is enhanced by at least 10%.

18. The method according to the claim 17 wherein response/level of IgG or IgM is enhanced by at least 10% for at least 14 days.

19. The method according to the claim 17 wherein response/level of IgG or IgM is enhanced by at least 10% for at least 21 days.

20. The method according to the claim 17 wherein response/level of IgG or IgM is enhanced by at least 10% for at least 28 days.

21. The composition or method according to claim 1 wherein when the pharmaceutical composition of Cannabidiol is administered with a Covid-19 vaccine, such administration of such composition is selected from any of the following:
i) before administering Covid-19 vaccine; or
ii) along with Covid-19 vaccine; or
iii) after administering Covid-19 vaccine; or
iv) before, along with and after administering Covid-19 vaccine.

22. A kit comprising a pharmaceutical composition comprising therapeutically effective amount of Cannabidiol and a Covid-19 vaccine.

23. A kit of claim 22 wherein a Covid-19 vaccine is selected from one or more of a component viral vaccine or one or more of a whole virus vaccine or any combination thereof.

24. Vaccine as claimed in claim 1 selected from m-RNA vaccine, adenoviral vector vaccine, inactivated vaccine, mixed vaccine having mRNA+recombinant+attenuated virus or any combination thereof.

25. The composition or method according to claim 11 wherein when the pharmaceutical composition of Cannabidiol is administered with a Covid-19 vaccine, such administration of such composition is selected from any of the following:
i) before administering Covid-19 vaccine; or
ii) along with Covid-19 vaccine; or
iii) after administering Covid-19 vaccine; or
iv) before, along with and after administering Covid-19 vaccine.

26. Vaccine as claimed in claim 11 selected from m-RNA vaccine, adenoviral vector vaccine, inactivated vaccine, mixed vaccine having mRNA+recombinant+attenuated virus or any combination thereof.
